(19) United States
(12) Patent Application Publication
(10) Pub. No.: US 2009/0187155 A1
(43) Pub. Date: Jul. 23, 2009

(54) MULTIFUNCTIONAL ARTICLES AND METHOD FOR MAKING THE SAME

(76) Inventor: Ali Razavi, Dallas, PA (US)

Correspondence Address: ZALE Patent Law, 121 Old Orchard Road, Clarks Summit, PA, 18503-2053, US

(21) Appl. No.: 12/301,356

(22) PCT Filed: Jul. 17, 2007
(86) PCT No.: PCT/US07/16194

§ 371 Date: Nov. 18, 2008

Related U.S. Application Data
(60) Provisional application No. 60/831,438, filed on Jul. 18, 2006.

(51) Int. Cl. A61F 13/15

(52) U.S. Cl. 604/367; 604/378

(57) ABSTRACT

An absorbent article (1000) includes “beads” (111) that are modified to add functional groups (113). The functional groups (113) add antimicrobial, fire retardant, alcohol repellant and other properties. In addition, the beads (111) may be inert diamond, or conductive graphite. The substrate (101) of the article (1000) may be made with the beads (111) and functional groups (113) retaining the properties. The beads (111) and functional groups (113) may be incorporated into super absorbent materials (117) and attached to the substrate (101), with or without a binder (103). The resulting article (1000) therefore may have antimicrobial, fire retardant, alcohol repellant and antistatic properties making it specifically useful in medical applications. It may also be made as a multi-layered article (1000) having different amounts of super absorbent (“SA”) particles (1207, 1307, 1407) printed or embedded in patterns on each layer (1100, 1200, 1300, 1400), thereby channeling liquids.

### CROSS REFERENCE TO RELATED APPLICATIONS

This application is a continuation-in-part of U.S. Provisional Patent Application “Biofunctional Articles for Personal Care Articles and Method for Making the Same” Ser. No. 60/831,438 filed Jul. 18, 2006 by the same inventor, Dr. Ali Razavi and claims priority from that application.

### BACKGROUND OF THE INVENTION

1. Field of the Invention

The present invention relates to woven and non woven fabrics and materials, having antimicrobial, absorbent, fire retardant, adsorbent, alcohol repellent and antistatic properties.

2. Discussion of Related Art

In many cases cloth and paper are used to absorb liquids. Many times the liquids are infected with microbes.

Super absorbent (“SA”) particles are well known materials which have been used in personal care articles, such as diapers. These particles are known to absorb several times their weight of water, saline solution, urine, blood and/or serous bodily fluids.

These materials have been described in numerous patents, such as U.S. Pat. Nos. 4,090,013, 4,693,713, 5,115,011, 5,578,318 and 6,433,058.

The liquid comes in contact where the SA particles are at the closest point to where the fluid enters the article, (the “entry point”).

The small amount of liquid saturates a small amount of SA particles near the entry point causing them to gel. The saturated SA particles expand as they gel. The gel acts as a barrier to the flow of additional liquids. Therefore, even though the remainder of the article has a great deal of dry SA particles; the liquid cannot go through the gel to reach the dry SA particles and leaks out of the article.

One attempt to correct this situation was to slow or reduce absorption of SA particles so that the fluid could reach more distant SA particles before being absorbed as described in U.S. Pat. No. 6,433,058. Such particles are prepared by contacting a substrate water-swellable, water-insoluble polymer particle with a polyvalent metal salt solution under conditions such that there is formed a polymer having an Absorption Rate Index of at least about 5 minutes.

Another patent, British Patent GB 2,280,115 A describes an absorbent article that contains coated SA particles at the entry point where body fluids are first released. The coating of the SA particles prevents swelling until the coating has dissolved in the body fluid or has been penetrated by it. These are SA particles that exhibit an activation time until swelling begins, which time can be varied by the coating's material and thickness. Some of the coating materials disclosed are non-reactive polysaccharides such as gelatin, microcrystalline cellulose and cellulose derivatives.

U.S. Pat. No. 5,115,011 addresses the problem of the gel blocking passage of additional liquids having an aqueous solution of two water soluble salts that come in contact with the gel. The first being a halogen, sulfate, acetate or nitrate of aluminum, calcium or magnesium, and the second being a monovalent metal salt or ammonium salt of at least one kind of an oxyacid selected from sulfurous acid and thiosulfuric acid. A dry blend of 0.6 g aluminum sulfate and 30 g polymer is prepared in Example for Comparison 3 of the patent, and is shown to have a blocking of 70 percent or more after 5 minutes.

U.S. Pat. No. 5,578,318 discloses the preparation of SA “hydrophobic coated particles” by dry blending materials, such as non-crosslinked polyacrylate salts, with a source of multivalent ions and, optionally, then adding an alcohol, certain wetting agents, and polysiloxane derivatives. The wetted material is dried prior to use. Example XXIII of this patent discloses a blend of 2.61 weight percent AQUALON A-250, 0.21 weight percent aluminum acetate, and 97.18 weight percent water.

U.S. Pat. No. 4,090,013 discloses materials prepared from a water-soluble anionic polyelectrolyte and a polyvalent metal cation source. However, the products are characterized in U.S. Pat. No. 5,578,318 as exhibiting gel blocking.

The above attempts were further complicated by the fact that as the SA particles absorbed liquids, they would expand and physically press against each other closing any liquid channels leading to deeper unsaturated SA particles.

In addition, if the liquids being absorbed are bodily fluids, there is always the problem of microbes. These can cause odor, infection and disease.

Even when anti-microbials are used they tend to be effective only where they are located. It is difficult to deposit them evenly on surfaces since they tend to migrate.

In the past many disposable materials are highly flammable. Being around electricity and electronic equipment, these have the potential to ignite. It would be desirable to have disposable materials which are fire resistant.

Also, since disposable materials are used on patients connected to sensitive equipment, static electricity buildup causes the equipment to give improper readings. It would be desirable to have anti-static materials.

Alcohol is used in many medical settings. If the materials used absorb the alcohol, they are saturated and cannot absorb the effluents from wounds, etc. It would be desirable to have a disposable material which is alcohol repellant.

Currently, there is a need for a material which will absorb liquids in a more efficient manner which will not exhibit gel blocking, resist microbial infections and have other desired characteristics.

### SUMMARY OF THE INVENTION

The present invention may be embodied as an absorbent article 1000 comprising:
a) A substrate 101;
b) A plurality of functional super absorbent particles (functional SAs) 110 incorporated in the substrate 101.
   Each of the functional SAs 110 is comprised of:
a) a super absorbent (SA) particle 117, and
b) a plurality of functional beads 115 attached to the SA 117.
   The functional beads 115 may be inert nanodiamond beads 111 having attached functional groups 113 which have antimicrobial, fire retardant group, alcohol repellant and antistatic properties.

The present invention may also be embodied as a super absorbent article for absorbing a liquid from a liquid entrance, comprising:

a plurality of layers each having a base material and super absorbent materials, the layers closer to the liquid entrance being the closer layers and layers away from the liquid entrance being deeper layers;

the closer layers allow liquids to more readily pass to deeper layers;

the deeper layers employ more super absorbent materials, more super absorbent material surface area and less ability to pass a liquid though them;

the invention described above wherein the super absorbent materials of each layer are attached to the base material according to a pattern; and

the patterns for the closer layers employ less surface area and less super absorbent materials compared with the deeper layers causing liquids to pass to the deeper layers providing more efficient absorption.

### OBJECTS OF THE INVENTION

It is an object of the present invention to provide a material which will absorb a larger amount of liquid without leaking as compared with prior art materials.

It is another object of the present invention to make the actual absorbent materials antimicrobial.

It is another object of the present invention to provide a material which more efficiently absorbs liquids.

It is another object of the present invention to provide a material which is fire retardant.

It is another object of the present invention to provide a material which is alcohol repellant.

It is another object of the present invention to make a material which exhibits anti-static properties.

It is another object of the present invention to provide a multi-layered material which has antimicrobial properties.

It is another object of the present invention to provide a material which can absorb body fluids and neutralize microbes.

It is another object of the present invention to provide a material which can absorb body fluids and resists infection.

It is another object of the present invention to provide a material which can absorb body fluids and resists odor.

It is another object of the present invention to evenly disburse anti-microbial materials where they are needed.

It is another object of the present invention to hold evenly disbursed anti-microbial materials where they were deposited.

### BRIEF DESCRIPTION OF THE DRAWINGS

A complete understanding of the present invention may be obtained by reference to the accompanying drawings, when considered in conjunction with the subsequent detailed description, in which:

FIG. 1 is a drawing illustrating the process of creating an absorbent article according to the present invention.

FIG. 2 is a drawing illustrating the process of creating an absorbent article according to another embodiment of the present invention.

FIGS. 3, 4, 5 show differing stages of completion of an absorbent article according to another embodiment of the present invention.

FIG. 6 is a perspective view of a multi-layered material according to one embodiment of the present invention.

FIG. 7 is a plan view of one embodiment of a pattern used on a first layer, closest to the liquid source.

FIG. 8 is a plan view of one embodiment of a pattern used on a second layer, just below the first layer.

FIG. 9 is a plan view of one embodiment of a pattern used on a third layer.

FIG. 10 is a plan view of one embodiment of a pattern used on a fourth layer.

FIG. 11 is a cross sectional view of the multi-layered material of FIG. 6.

FIG. 12 is an illustration of the major parts of a flatbed laminating device useful with the present invention.

FIG. 13 shows the functional bead with attached antimicrobial functional groups.

### DETAILED DESCRIPTION OF THE PRESENT INVENTION

There is a need for a superabsorbent articles to collect undesirable liquids such as effluents from a patient's wound or a baby's dirty diaper.

These also include microbes which potentially are pathogenic and can cause additional infection and odor. In the prior art, there are articles which have antimicrobial surface properties and other articles which have superabsorbent properties. The idea is to capture the effluents, and hold them adjacent to antimicrobial particles to kill any microbes.

This does not occur in an efficient manner if the superabsorbent and antimicrobial are not evenly applied and are in close proximity to each other. Conventional methods do not effectively achieve this goal.

The present invention attaches the antimicrobials directly to the superabsorbent particles, then attaches the superabsorbent particles to a substrate.

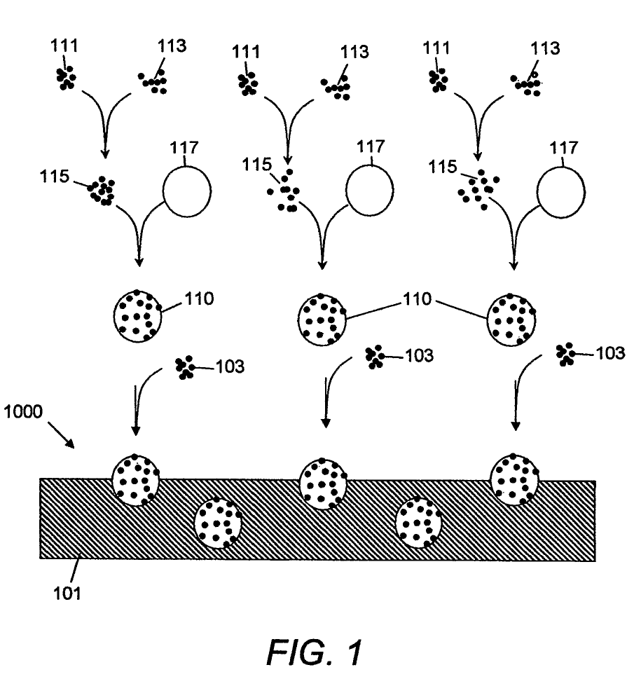

In FIG. 1 a generalized process is shown for implementing the present invention. A bead 111 which is typically an inert carrier particle is modified to attach a functional group 113. The functional groups 113 cause the inert bead 111 to exhibit a certain functional characteristic. The functional groups 113 may be antimicrobial groups, fire retardant groups, or alcohol repulsion groups which kill microbes, minimize burning or repel alcohol, respectively. Many of these are covalently attached to the carrier beads 111 giving them a function.

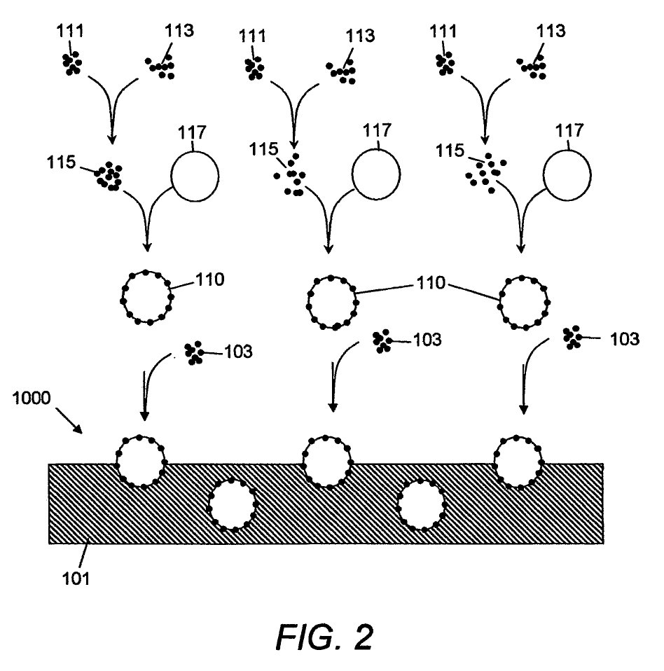

The beads 111 with attached functional groups are referred to as a “functional beads” 115. In order to insure that the right concentration of functional beads 115 are adjacent the absorbed liquids, the functional beads 115 are attached to a suberabsorbent (SA) particle 117 to create a plurality of functional SAs 110. In FIG. 1 they are physically mixed at the proper proportions and in FIG. 2, the SAs 117 are coated with functional beads 115.

The functional SAs 110 are then sprayed to attach to the surface of a substrate 101. Alternatively, they are shot with an electrostatic gun into the substrate 101 to embed into the substrate 101.

In an optional embodiment, a binder may be used to attach the functional SA 110 to the substrate 101. A binder may be thermoplastic or thermoset or U curable materials can be mechanically mixed and applied to the surface of the functional SA 110 or the substrate 101 prior to applying the functional SA 110 to substrate 101.

The functionalized SA 110 can be fused or mixed or alternatively covalently bonded with thermoset or thermoplastic resin materials (a binder 103) and then applied as a top coat to a substrate 101 comprised of fibers, fabrics, webs, batts and single or multi-layer nonwoven constructions, employed in manufacturing of wound dressing, medical drapes, surgical gowns, surgical masks disposable diapers, filter media and others.

The activated binder materials 103 can fuse the functional SAs 110 to the substrate 101 by UV radiation, heat or catalytic processes.

The functional SAs 110 can be deposited on fibers or fabrics by varieties of techniques including but not limited to: electromagnetic brush as described in US Patent Application No. 2005/0202164. They may also be attached electrostatically. Other methods, such as fluidized bed coating methods or thermal or flame spraying, may also be used.

Functional SAs 110 can be deposited to surface of any desired fabric in forms of continuous or selective nano-fibers by an electro-spinning process.

The beads 111 are preferably comprised of nanodiamonds.

Nanodiamonds are a unique form of matter, comprised of carbon containing a compressively stressed rigid diamond core with a chemically tunable strained graphitic shell. These are produced in accordance with U.S. Pat. Nos. 5,861,349 and 5,916,955, utilizing the controlled detonation synthesis of carbon precursor. This results in the creation of particles with diameters of 4-6 nanometers (4-6×10⁻⁹ meters) and a tremendously large surface area with bioavailability (460 m²/gm). Uniquely structured Nanodiamond particles yield the intrinsic properties of diamond, including mechanical strength, thermal conductivity and chemical stability. This Nanodiamond carbon materials with non-toxic, inert is in a capsule that can be chemically modified to produce tunable surface functionality ideal for bio-functional novel materials for intended applications. The short list of these nanoparticles along with diamoned percentages available are listed below:

| Nanodiamond | |
|---|---|
| particles | Diamond, % wt. |
| UD50 | <30 |
| UD90 | 70-81 |
| UD98 | 74-85 |
| Oxidized UD90 | 92-95 |
| Oxidized UD98 | 92-95 |
| Oxidized UD50 | 95-96.5 |
| Diamond (>5 μm) | 97-97.5 |
| HOPG (Graphite) | 0 |

### Anti-Microbials

Bioactive functional group 113 attached to the beads 111 are used to provide resistance to microbes such as bacterial, fungal and viral injections and combinations of such infections. Several useful bioactive functional groups are listed below:

Potassium peroxymonosulfate, Keflex® (cephalexin), phthalimides, acetamides, phthalonitriles, hydroxy benzoates, isothiazolinones, nitropropane diols, carbamates, methyl ureas, benzimidazoles, salicylanilides, mercury acetates, organozinc compounds, metals such as silver, copper and zinc, and ions of such metals.

Among the liquid anti-microbial agents that are suitable in certain applications, a preferred anti-microbial agent is dibromocyanoacetamide (for example, Amerstat® 300 made by Drew Industrial Division of Ashland Chemicals, Boonton, N.J. 07005).

In addition, solid anti-microbial agents that are preferred include 2-bromo-2-nitropropane-1,3-diol (for example, Canguard® 409 made by Angus Chemical Co., Buffalo Grove, Ill. 60089) and 3,5-dimethyltetrahydro-1,3,5-2H-thiazine-2-thione (for example, Nuosept® S made by Creanova, Inc., Piscataway, N.J. 08855 or Troysan®. 142 made by Troy Chemical Corp., West Hanover, N.J. 07936).

Other solid anti-microbial agents include N-(trichloromethyl)-thiop-hthalimide (for example, Fungitrol®. 11 made by Creanova, Inc.), butyl-p-hydroxy-benzoate (for example, Butyl Parabens®. made by International Sourcing Inc., Upper Saddle River, N.J. 07458), diiodomethyl-p-tolysulfone (for example, Amical®. WP made by Angus Chemical Co.), and tetrachloroisophthalonitrile (for example, Nuocide® 960 made by Creanova, Inc.).

Metals such as silver, copper and zinc and their metal ions also have anti-microbial properties. Silver ions have widespread effect as an anti-microbial agent. For example, silver ions may be effective against bacteria such as Escherichia coli and Salmonella typhimurium, and mold such as Asperigillus.

Sources of silver for functional groups for anti-microbial use include metallic silver, silver salts and organic compounds that contain ionic silver. Silver salts may include for example, silver carbonate, silver sulfate, silver nitrate, silver acetate, silver benzoate, silver chloride, silver fluoride, silver iodate, silver iodide, silver lactate, silver nitrate, silver oxide and silver phosphates. Organic compounds containing silver may include for example, silver acetylacetonate, silver neodecanoate and silver ethylenediaminetetraacetate in all its various salts.

Silver containing zeolites (for example, AJ10D containing 2.5% silver as Ag(I), and AK10D containing 5.0% silver as Ag(I), both made by AgION™. Tech. L.L.C., Wakefield, Mass. 01880) are of particular use. Zeolites are useful for functional groups because when carried in a polymer matrix they may provide silver ions at a rate and concentration that is effective at killing and inhibiting microorganisms without harming higher organisms.

Silver containing zirconium phosphate (for example, AlphaSan® C 5000 containing 3.8% silver provided by Milliken Chemical, Spartanburg, S.C. 29304) is also particularly useful. In general zirconium phosphates act as ion exchangers. However, AlphaSan® C 5000 is a synthetic inorganic polymer that has equally spaced cavities containing silver, wherein the silver provides the anti-microbial effects. Silver zirconium phosphates are typically incorporated into powder coatings between 0.1 and 10 percent by weight and particularly 0.5 to 5 percent by weight of the total powder coating formulation.

### Super Absorbent Particles

Superabsorbent polymeric powders (SA 117) suitable for use in the present invention include, but are not limited to, a wide variety of anionic, cationic, and nonionic materials. Suitable polymers include polyacrylamides, polyvinyl alcohols, ethylene maleic anhydride copolymer, polyvinylethers, polyacrylic acids, polyvinylpyrrolidones, polyvinylmorpholines, polyamines, polyethyleneimines, polyquaternary ammoniums, natural based polysaccharide polymers such as carboxymethyl celluloses, carboxymethyl starches, hydroxypropyl celluloses, algins, alginates, carrageenans, acrylic grafted starches, acrylic grafted celluloses, chitin, chitosan, and synthetic polypeptides such as polyaspartic acid, polyglutamic acid, polyasparagins, polyglutamines, polylysines, and polyarginines, as well as the salts, copolymers, and mixtures of any of the foregoing polymers.

It is also contemplated that the substrate can be made of foamed starting materials such as fibers and sheets and foamed powders can further improve fluid absorbency due to an increase in respective surface areas. In the instance of foamed sheets, it is noted that above-mentioned U.S. Pat. No. 5,985,434 utilizes a water-swellable, water insoluble polymeric foam having an average cell size between about 10 microns to about 100 microns with about 10 to about 50 microns being preferred. It is also contemplated that foamed fibers can be used as a polymeric material to be coated. Foaming of polymeric materials such as sheets, films, fibers, etc., creates even more surface area for coating and absorbency and thus the respective particle sizes of the powder and cells should be compatible.

Foamed polymeric materials can be present in the absorbent foam in a weight amount that is between about 50 wt % to 100 wt %, beneficially between about 60 wt % to about 100 wt %, more beneficially between about 70 wt % to about 100 wt %, suitably between about 80 wt % to about 100 wt %, more suitably between about 90 wt % to about 100 wt %, and even more suitably between about 95 wt % to about 100 wt %, wherein all wt % s are based on the total weight amount of the polymer, crosslinking agents, and any other optional components present in the absorbent foam.

In one embodiment, it is desired that the absorbent foam consist essentially of the polymer and, optionally, any crosslinking agent used to crosslink the polymer.

### Curable Resins Binders:

A variety of curable resins may be used as binder 103 including epoxies, saturated and unsaturated polyesters, polyester-epoxy hybrids, acrylics, and mixtures thereof may be utilized in the invention.

When heated above their respective curing temperatures or exposed to radiation curing, as the case may be, curable resins flow to form a coating. When the resin cures, superabsorbent polymeric powders that are proximate to the curable resins become stably adhered to the coating. Thus, when curable resin powders and superabsorbent polymeric powders are coated onto a surface of a substrate, such as polymeric sheets or fibers, the above-mentioned curing results in particles of superabsorbent polymeric powders becoming stably adhered to the substrate. When curing occurs, the superabsorbent polymeric powders do not cure or melt and thus remain as discrete particles in the coating.

Radiation curable resins may also be used as a binder 103 including unsaturated polyester resins along with vinyl ether or an acrylate crosslinker and a photoinitiator.

Curable resinous powders that cure at temperatures below about 300.degree F. are well established. The above-mentioned epoxy, polyester, polyester-hybrid, acrylic, and admixtures thereof resins utilize curing agents and/or catalysts capable of obtaining curing temperatures on the order of 300 degree F.

U.S. Pat. No. 5,270,416 also discloses glycidyl methacrylate containing resins crosslinked with carboxylic acid functional crosslinkers and polyesters. If acrylic resins are used, GMA resins such as PD 7690 from Anderson Development Company can be used with DDA as curing agent in presence of catalysts that promote this reaction. Crosslinkers may comprise aliphatic dicarboxylic acid.

U.S. Pat. Nos. 4,147,737 and 5,168,110 disclose other glycidyl functional crosslinkers that can be used with acid functional polyesters as thermosetting powder coating compositions. Epoxy resins such as that are based on bisphenol A can also be used as crosslinkers to form hybrid powder coatings

### Catalysts

In all of the above compositions, suitable catalysts can be used to enhance low temperature cure characteristics of the binder materials. For all of the thermosetting compositions involving acid functional and glycidyl functional materials suitable catalysts can be chosen from amines (such as DBU), ammonium salts (such as tetra butyl ammonium bromide, benzyl trimethyl ammonium chloride), phosphine (such as triphenyl phosphine), phosphonium salts (such as ethyl triphenyl phosphonium bromide), imidazole (such as 2-methyl imidazole, 2-phenyl imidazole), imidazole adducts (such as P101 from shell, HT 3261 from Ciba Geigy) can be used. Examples of catalyst that are discussed in these patents are compounds containing amine, phosphine, heterocyclic nitrogen, ammonium, phosphonium, arsonium or sulfonium moieties. Especially preferred are the alkyl-substituted imidazoles; 2,5-chloro-4-ethyl imidazole; and phenyl substituted imidazoles, and mixtures thereof. Even more preferred are 2-methyl imidazole; 2-ethyl, 4-methyl imidazole; 1,2-dimethylimidazole; and 2-phenyl imidazole. Especially preferred is 2-methyl imidazole. Particularly suitable catalysts are those quaternary phosphonium and ammonium compounds such as, for example, ethyltriphenylphosphonium chloride, ethyltriphenylphosphonium bromide, ethyltriphenylphosphonium iodide, ethyltriphenylphosphonium acetate, ethyltriphenylphosphonium diacetate (ethyltriphenylphosphonium acetate acetic acid complex), ethyltriphenylphosphonium tetrahaloborate, tetrabutylphosphonium chloride, tetrabutylphosphonium bromide, tetrabutylphosphonium iodide, tetrabutylphosphonium acetate, tetrabutylphosphonium diacetate (tetrabutylphosphonium acetate acetic acid complex), tetrabutylphosphonium tetrahaloborate, butyltriphenylphosphonium tetrabromobisphenate, butyltriphenylphosphonium bisphenate, butyltriphenylphosphonium bicarbonate, benzyltrimethylammonium chloride, benzyltrimethylammonium hydroxide, benzyltrimethylammonium tetrahaloborate, tetramethylammonium hydroxide, tetrabutylammonium hydroxide, tetrabutylammonium tetrahaloborate, and mixtures thereof

### Application to Substrate

The biofunctional beads 115 may be attached to the substrate 101, or covalently or mechanically incorporated into the absorbent materials including but not limited to the absorbent structures, absorbent binder structure with above characteristics. These can be selectively deposited on the fabric (substrate) of choice.

One such method of attaching particles to the substrate is electrostatic powder coating of the substrate.

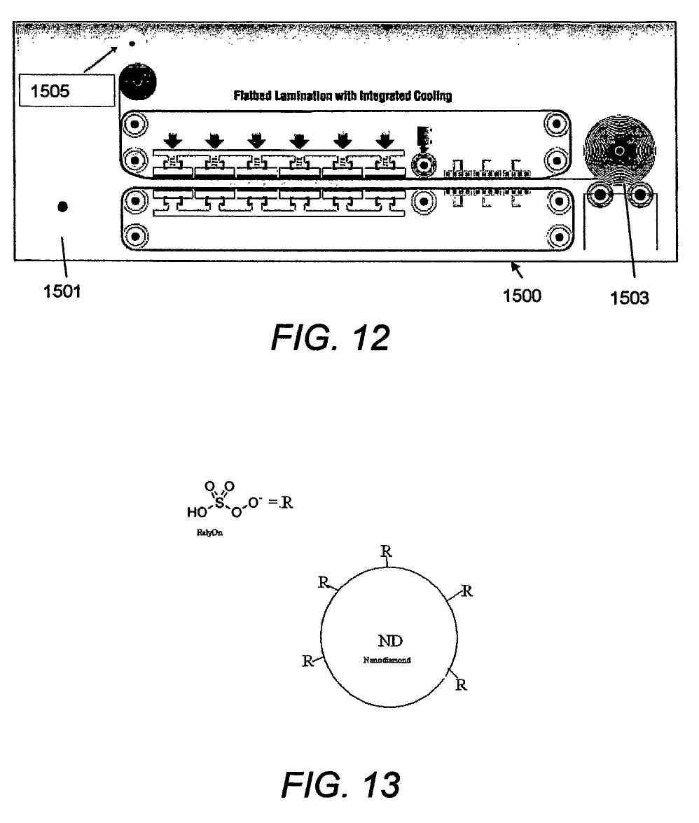

Another method is a flatbed lamination process. The flatbed lamination process may be performed by a device such as a Glenro SCA-72 powder adhesive coater. FIG. 12 shows such a device 1200.

A substrate roller 1501 holds substrate to be processed. A hopper (not shown) drops powder to be attached to the substrate onto the unrolled substrate.

A power adhesive, or an adhesive web form adhesive roller 1505 are placed on the substrate. The substrate is then heated to cause adhesion of the powder to the substrate then cooled.

Once cooled the substrate is taken up on take up roller 1503. The rollers can then be reversed to coat the opposite side of the substrate.

Particles may be attached to the substrate in patterns in order to adjust the amount of adsorption. It may be patterned to provide flow channels, regions of higher and lower fluid intake and absorption dams for preventing fluid leakage and other desired features including but not limited to manufacturing of extremely thin absorbent article.

As stated above, SA particles are readily available. U.S. Pat. No. 4,693,713 discloses an absorbent for blood and serous bodily fluids, the absorbent comprising a physical mixture of certain particles and certain compounds. The compounds are described as water soluble, present in the form of a pourable powder at ambient temperature, and not harmful to health. The patent teaches that the compound may be added to the polymer by dissolving it in the monomer solution, or that the compound can be added to the polymer preparation process at any time in dry or dissolved form. Dry blends of polymer and compound are prepared in the examples of the patent.

Oligodynamic iontophoresis is capable of reducing bacterial colonization fifteen to one-hundred fold and can be produced on super absorbent and any other fabric incorporated in personal care products as well. This can be done by combination of technologies including but not limited to mechanical mixing of base materials with graphite or others, to provide conductor paths, followed by silver nano-deposition in order to provide iontophoresis effect.

The mechanical mixing of silver metal, silver alloy, graphite, ZnO, Zeolite, Silversulfadizine and other suitable materials with super absorbent materials by conventional mechanical mixing will provide multifunctional composite articles. These may be mixed in differing ratios to provide different adsorption and antimicrobial effects.

At the same time, other materials of interest such as anti-rash, deodorizing and others can be mechanically or otherwise mixed with super absorbent materials in order to provide personal care articles with specific bio-functional products.

The Zeolite, silver particles and other particles may be printed on the base material. The desired materials are provided in a solvent-based or water-based dispersion which can be deposited by gravure, screen, dot, pad, and flexographic & electrophoretic technology on the personal care fabrics. This provides biofunctional products for personal care applications which are not presently available.

FIG. 2 is similar to FIG. 1 except that the functional beads 115 are attached to the surface of SA 117 as opposed to being embedded in the SA 117.

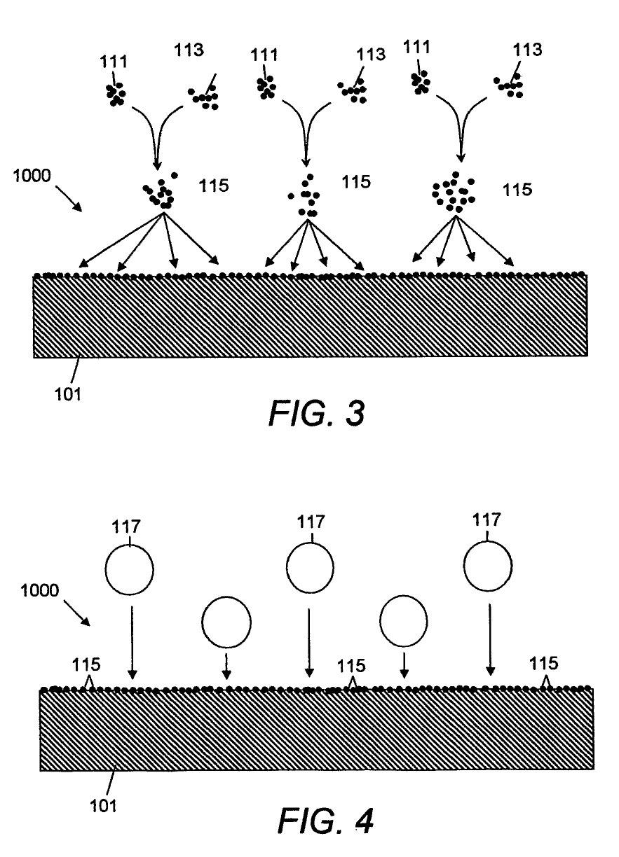

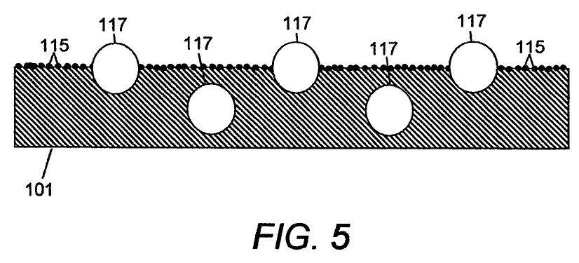

FIGS. 3-5 show the process of directly attaching functional beads 115 to the surface of substrate 101. SAs 117 are then attached to the surface of substrate 101. Optionally, SAs 117 may be embedded into substrate 101.

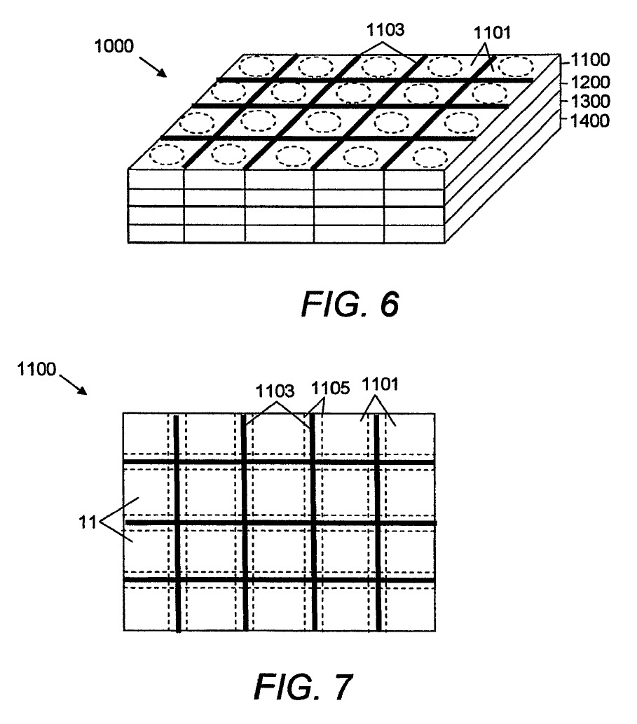

FIG. 6 is a perspective view of a multi-layered material according to one embodiment of the present invention. FIG. 6 is an absorbent article 1000 according to the present invention that provides increased absorption properties, having several layers 1100, 1200, 1300, and 1400 each made of a substrate 1101. Substrate 1101, as stated above, may be a non-woven cellulosic material, cotton or other fiber, or a material woven from threading. A liquid can pass through substrate 1101 by capillary action and wicking action.

Substrates 1101, 1201, 1301 and/or 1401 may be treated with any of a number of known functional groups mentioned above.

The super absorbent particles (SAs 117 of FIGS. 1, 2, 4, 5) may be attached to, or embedded in the substrate 1101 in a pattern. The pattern shown in FIG. 6 is a regular grid 1103. The SA particles may be created using any of a number of known technologies, such as that described in U.S. Pat. No. 4,693,713. The SA particles may also be modified to delay their absorption as described above. This activation period before absorption should be at a minimum five minutes. Preferably it should be no more than 60 minutes. This allows fluids to disperse throughout the article 1000 before absorption begins.

The square grid pattern 1103 employing SA particles are either printed upon, or embedded into the substrate 101 of layer 1100. Liquids which impinge upon the SA polymer gridlines 1103 are absorbed by the SA particles and expand to the dashed lines indicated by expanded grid 1105.

The portions between the grid lines 1103 are base material 1101 which provides drainage channels for liquid transportation along or through layer 1100.

The absorbent article 1000 exhibits the advantage of providing different absorption rates in each layer 1100, 1200, 1300, 1400 based upon the amount of SA polymer and the surface area of the SA particles. The absorbency differential can be provided by using different patterns employing differing amounts and particle sizes of SA materials. These patterns may be created by many commercial processes including but not limited to gravure, screen, dot, pad, and flexographic and electrophoretic printing technologies.

Alternatively, super absorbent materials could be dissolved in proper solvent and its solution can be cast in pattern format as described in U.S. Pat. No. 5,578,318 Honeycutt.

Layers 1100, 1200, 1300, and 1400 provide a thin, effective multi-level liquid filtration device, in which each layer of stack is composed of SA materials arranged in specific pattern format including but not limited to dots, lines, triangles and any other desirable geometry.

FIG. 7 is an example of the regular grid pattern 1103 used for layer 1100. Dashed lines 1105 show the expanded grid 1105 after absorbing fluids.

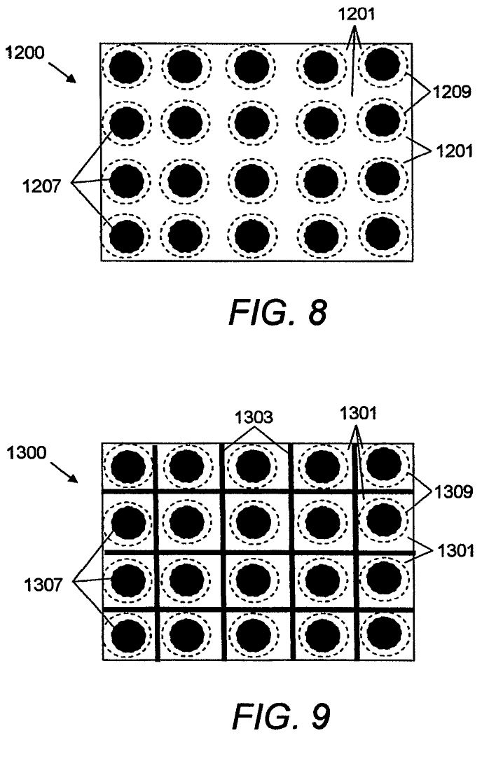

Similarly, FIG. 8 employs a pattern of circular super absorbent regions 1207 for layer 1200. The super absorbent regions 1207 expand to the dashed perimeters 1209 after absorbing fluids.

FIG. 9 shows a pattern used in layer 1300. Here another regular grid 1303 is employed with another pattern of circular regions 1307 centered within each grid square. This reduces the size of spacing between the two patterns.

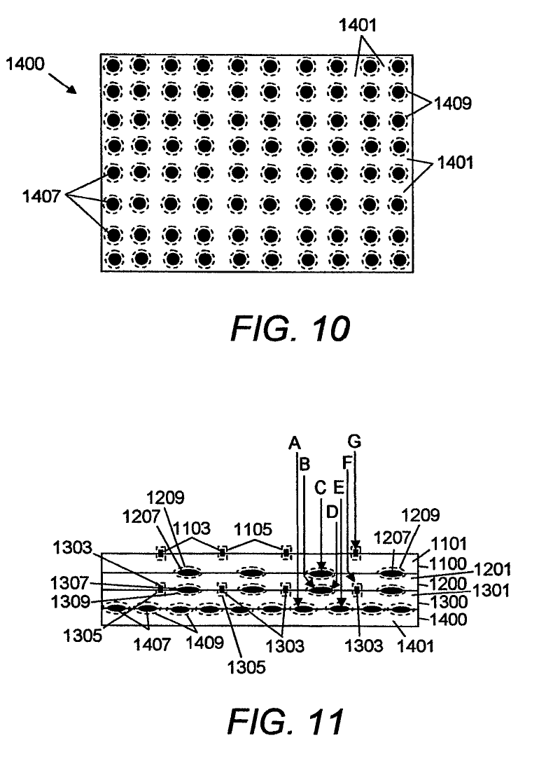

FIG. 10 shows that layer 1400 employs a pattern 1407 of circular regions each being much smaller than that of the other layers. This provides significantly more SA particles and a much greater surface area. It also provides much smaller spacing between circular regions 1407 reducing the wicking through layer 1400.

The optimum design of pattern dimensions will take into consideration the volume expansion that SA particles experience upon liquid exposure in each layer 1100, 1200, 1300, and 1400. Therefore, each layer 1100, 1200, 1300, and 1400 provides certain absorbency capacity with specific channel openings to transfer extra liquid to the additional layer, one at a time.

FIG. 11 shows a cross section view of the embodiment of the article shown in FIGS. 6-10. Here layer 1100 is shown with grid pattern 1103 made of partially embedded SA particles. Please note that even when the SA particles expand as shown by the dashed lines marked 1105, there is a significant amount of surface area of base material 1101 for liquids to pass through as indicated by arrows A-G.

Layer 1200 is located directly under layer 1100 and receives liquids which pass through layer 1100. This employs a regular square grid 1203 and a second pattern of disk-shaped regions 1207. As it is shown, as the square grid 1203 and the disk patterns 1207 expand, the channels between them 1211 become smaller and reduce and direct flow past layer 1200 to layer 1300.

Arrows A and E show channels for liquids passing through layer 1100-1300 to be absorbed by SA polymer region 1407 in layer 1400. Similarly, arrows B and D show liquids passing through layers 1100 and 1200 to be absorbed by SA region 1307 in layer 1300. Arrow C shows a liquid passing through layer 1100 to be absorbed by SA polymer region 1207 in layer 1200. Similarly, arrow G shows a liquid being immediately absorbed by SA polymer region in the regular gridlines 1103 of layer 1100.

Below are a summary of the various embodiments of the present invention.

1. Functional groups 113 are attached directly to a substrate 101.

2. Functional groups 113 are attached directly to super absorbents 117 to create a functional super absorbent 110 that is attached to a substrate 110.

3. Functional groups 113 are attached to beads 111 to make functional beads 115 that are attached to super absorbents 117 to create functional super absorbents 110 which are attached to a substrate 101.

4. The embodiments 1, 2, or 3 above employing a binder

5. Functional groups 113 are attached directly to a binder 103.

### Biofunctional Threads

The functional beads can be incorporated covalently in lattice (monomer) unit structure of resin materials before converted to fibers for making the substrate. Such fibers provide platform for manufacturing of useful substrates such as fabrics, webs, single/multi-layer, woven/non-woven construction, which are employed in manufacturing of wound dressing, medical drapes, surgical gowns, surgical masks, disposable diapers, and filter media.

In another alternative embodiment, the functional groups may also be attached covalently in lattice (monomer) unit structure of resin materials before converted to fibers for making the substrate.

### Binder

Functional beads can be chemically incorporated covalently in the molecular chain structure of the binder materials including but not limited to thermoplastic or thermoset or UV-curable or other prior art binders.

Alternatively, the nano-silver (as described above) can be deposited on any fabric incorporated in the design of personal care articles for the same property enhancements. This may be accomplished by coating fibers of substrates 101, 1101, 1201, 1301 and 1401 with antimicrobial nano-materials listed above. The fibers may then be pressed into a non-woven base material in a desired pattern format.

Melt extruded fibers suitable for forming the non-woven fibrous layer or webs of the present invention non-woven cleansing articles or wipes can be produced from a wide variety of thermoplastic polymers that are known to form fibers.

Suitable thermoplastic polymers are selected from polyolefins, polyamides, polyesters, copolymers containing acrylic monomers, and blends and copolymers thereof. Suitable polyolefins include polyethylene, e.g., linear low density polyethylene, high density polyethylene, low density polyethylene and medium density polyethylene; polypropylene, e.g., isotactic polypropylene, syndiotactic polypropylene, blends thereof and blends of isotactic polypropylene and atactic polypropylene; and polybutylene, e.g., poly(1-butene) and poly(2-butene); polypentene, e.g., poly-4-methylpentene-1 and poly(2-pentene); as well as blends and copolymers thereof. Suitable polyamides include nylon 6, nylon 6/6, nylon 10, nylon 4/6, nylon 10/10, nylon 12, nylon 6/12, nylon 12/12, and hydrophilic polyamide copolymers such as copolymers of caprolactam and an alkylene oxide, e.g., ethylene oxide, and copolymers of hexamethylene adipamide and an alkylene oxide, as well as blends and copolymers thereof.

Suitable polyesters include polyethylene terephthalate, polybutylene terephthalate, polycyclohexylenedimethylene terephthalate, and blends and copolymers thereof. Acrylic copolymers include ethylene acrylic acid, ethylene methacrylic acid, ethylene methylacrylate, ethylene ethylacrylate, ethylene butylacrylate and blends thereof. Particularly suitable polymers are polyolefins, including polyethylene, e.g., linear low density polyethylene, low density polyethylene, medium density polyethylene, high density polyethylene and blends thereof, polypropylene; polybutylene; and copolymers as well as blends thereof.

As used herein, the term “fiber” includes fibers of indefinite length (e.g., filaments or spunbond fibers) and fibers of discrete length, e.g., staple fibers or meltblown fibers. The extruded fibers used in connection with the present invention may be multi-component fibers where one or more of the components can contain one or more lathering surfactants. The term “multi-component fiber” refers to a fiber having at least two distinct longitudinally coextensive structured polymer domains in the fiber cross-section, as opposed to blends where the domains tend to be dispersed, random, or unconstructed. The distinct domains may thus be formed of polymers from different polymer classes (e.g., nylon and polypropylene) or can be formed of polymers from the same polymer class (e.g., nylon) but which differ in their properties or characteristics. The term “multi-component fiber” is thus intended to include, but is not limited to, concentric and eccentric sheath-core fiber structures, symmetric and asymmetric side-by-side fiber structures, island-in-sea fiber structures, pie wedge fiber structures, and hollow fibers of these configurations. Different polymers can be used to provide different properties or used as carriers for different melt additive components or additives.

### Alcohol Repellency

Functional beads (115 of FIGS. 1-5) with or without SAs 117 can be covalently attached to moieties including but not limited to, fluorinated urethane derivates, fluorocarbons or silicones. These produce lower surface energies which can be incorporated in fabric materials for alcohol repellency applications.

Scientific Anglers Protective Material is PM 870 from the 3M Company is a C-4 based polymer which exhibits alcohol repellency properties that may be used as the substrate. The functional groups 113, beads 111, and super absorbents 117 may be used with these materials to add alcohol repellant capabilities to its other properties.

### Anti-Static

Functional beads (115 of FIGS. 1-5) can be produced with majority of its structure composed of graphite which conducts electricity. This can provide electrostatic dissipation when used as the beads.

The functional beads (115 of FIGS. 1-5) with alcohol repellency and electrical conductivity can also be incorporated covalently in lattice (monomer) unit structure of resins materials, before converted to fibers.

Functional beads (115 of FIGS. 1-5) with proper graphitic conductive phase can be chemically incorporated within unit structure of the alcohol repellent materials in prior arts in providing repellency and electrical conductivity for static decay properties.

### Fire Retardant

Functional beads can be covalently attached to moieties including but not limited to organic, inorganic, organo-metallic or intumescent fire retardant additives in order to provide fire resistance or retardant additives.

One such fire retardant powder is FR- (Griltex D1990C P1) from EMS-CHEMIE (North America) Inc. Griltex D 1990C was evaluated via a vertical burn test (underwriter Laboratories No. 94). The results were a V-o rating indicating that the material was self extinguishing. Incorporating using these materials as the functional groups will provide fire retardant characteristics to the manufactured article.

### Adsorption Materials

Functional beads 115 can be fused or covalently attached to metal oxides to adsorb gases. These include MgO, CaO, TiO₂, ZrO₂, FeO, V₂O₃, V₂O₅.Mn₂O₃, Fe₂O₃, NiO, CuO, Al₂O₃, ZnO and mixtures thereof.

The substrate, absorbent binder structure including but not limited to water soluble ionic polymers with radiative crosslinking capabilities (as described in U.S. Pat. No. 6,964,803), without radiative crosslinking (U.S. Pat. No. 6,887,961) cellulose fiber fluff based materials (U.S. Pat. No. 5,387,385), foamed polymeric, foamed composite (U.S. Pat. Nos. 6,241,713 & 5,720,832), crosslinkable binder with particles (U.S. Pat. No. 6,822,135), water-swellable polymers with phase-separating elastomeric, (U.S. Pat. No. 7,049,000) absorbent with lotion (U.S. Pat. No. 7,060,867), hydrophilic meltspun fabric (U.S. Pat. No. 5,901,706), absorbent composite with highly crosslinked superaborbent polymer (U.S. Pat. No. 6,720,073), binder treated particles (U.S. Pat. No. 6,391,453), EP 0631768 A1.

Different combinations of functional groups may be used to provide different capabilities. Several are listed below.

Functional beads+Silver ions or metal+nano-sorption (metal oxides)+binder can be attached to the fibers or fabric of the choice, in providing adsorption and killing microorganisms.

Functional beads+Silver ions or metal+nano-sorption (metal oxides) can be embedded to fibers or fabrics of the choice in providing adsorption and killing of microorganism.

### Test Results

The following antimicrobial testing was performed on non-woven fabric based on ASTM E2149-01 procedures.

Nano-diamond beads were covalently attached to Rely-on broad spectrum disinfectant with active ingredient Potassium peroxymonosulfate (U.S. Pat. No. 4,822,512). The functional beads with Rely-on are shown in FIG. 13. This material subsequently were bonded with D1582P1 made by EMS-Griltech (a unit of EMS-Chemical North America) powder and then electrostatically applied to the fabric using a Nordson Versa-Spray Gun. This coating subsequently fused to the fabric material at 80 degrees Centigrade. The operating voltage were at 60 DC voltage for this coating designated as material B.

Antimicrobial testing was performed on fused nano-diamond with Rely-on+D1582P1. The results are listed below:

| | Organism Count (CFU/ml) | | | |
|---|---|---|---|---|
| | Sample | | Time = t₀ + | Percent |
| Organism | Identification | Time = t₀ | 8 hours | Reduction |
| Klebsiella | A-6 | 1.42 × 10⁵ | <1 × 10 | 99.99% |
| Pneumoniac | | | | |
| ATCC-4652 | | | | |
| Staphylococcus | A-6 | 1.1 × 10⁵ | 1 × 10 | 99.99% |
| Aureus | | | | |
| ATCC 6538 | | | | |

While several presently preferred embodiments of the novel invention have been described in detail herein, many modifications and variations will become more apparent to those skilled in the art.

What is claimed is:

1. An absorbent article 1000 comprising:
a) A substrate 101;
b) A plurality of functional super absorbent particles (functional SAs) 110 incorporated in the substrate 101.

2. The absorbent article 1000 of claim 1 wherein each of the functional SAs 110 are comprised of:
a) a super absorbent (SA) particle 117, and
b) a plurality of functional beads 115 attached to the SA 117.

3. The absorbent article 1000 of claim 1 wherein each of the functional SAs 110 are comprised of:
a) a super absorbent (SA) particle 117, and
b) a functional group 113.

4. The absorbent article 1000 of claim 2 wherein the functional beads 115 are inert beads 111 having attached functional groups 113.

5. The absorbent article 1000 of claim 3 wherein the functional groups 113 are covalently attached to a material which is used to create a thread used to weave the substrate 101.

6. The absorbent article 1000 of claim 1 wherein the substrate 101 is a non-woven material.

7. The absorbent article 1000 of claim 1 further comprising:
a binder material 103 used to attach the functional SAs 110 to the substrate 101.

8. The absorbent article 1000 of claim 2 wherein the attached functional group 113 is an antimicrobial group.

9. The absorbent article 1000 of claim 2 wherein the attached functional group 113 is a fire retardant group.

10. The absorbent article 1000 of claim 2 wherein the inert beads 111 are partially diamond.

11. The absorbent article 1000 of claim 2 wherein the inert beads 111 are partially graphite.

12. The absorbent article 1000 of claim 2 wherein the inert beads 111 are about 5 microns in diameter.

13. The absorbent article 1000 of claim 3, wherein the functional group 113 is selected from at least one of the group consisting of:
Potassium peroxymonosulfate, cephalexin, phthalimides, acetamides, phthalonitriles, hydroxy benzoates, isothiazolinones, nitropropane diols, carbamates, methyl ureas, benzimidazoles, salicylanilides, mercury acetates, organozinc compounds, silver, copper and zinc, dibromocyanoacetamide, 2-bromo-2-nitropropane-1,3-diol, 3,5-dimethyltetrahydro-1,3,5-2H-thiazine-2-thione, N-trichloromethyl)-thiophthalimide, butyl-p-hydroxy-benzoate, diiodomethyl-p-tolysulfone and tetrachloroisophthalonitrile.

14. The absorbent article 1000 of claim 1, wherein the substrate 101 is formed from at least one of the group consisting of:
polyolefins, polyamides, polyesters, copolymers containing acrylic monomers, polyethylene, polypropylene, polybutylene, polyamides, hydrophilic polyamide copolymers, alkylene oxide, hexamethylene adipamide and an alkylene oxide.

15. The absorbent article 1000 of claim 2, wherein the super absorbents 117 are formed from at least one of the group consisting of:
polyacrylamides, polyvinyl alcohols, ethylene maleic anhydride copolymer, polyvinylethers, polyacrylic acids, polyvinylpyrrolidones, polyvinylmorpholines, polyamines, polyethyleneimines, polyquaternary ammoniums, natural based polysaccharide polymers and synthetic polypeptides.

16. The absorbent article 1000 of claim 2, wherein a flatbed lamination process is used to attach particles to the substrate 101.

17. An absorbent article 1000 for absorbing a liquid from a liquid entrance, comprising:
a) a plurality of layers 1100, 1200, 1300, 1400 each having a substrate 1101, 1201, 1301, 1401 and super absorbent materials 1207, 1307, 1407, the layers closer to the liquid entrance being the closer layers 1100, 1200 and layers away from the liquid entrance being deeper layers 1300, 1400;
b) the closer layers 1100, 1200 allow liquids to more readily pass to deeper layers;
c) the deeper layers 1300, 1400 employ more super absorbent materials 1207, 1307, 1407, more super absorbent material surface area and less ability to pass a liquid though them.

18. The absorbent article 1000 of claim 17 wherein the super absorbent materials 1207, 1307, 1407 of each layer are attached to the substrate 1201, 1301, 1401 according to a pattern; and
the patterns for the closer layers employ less surface area and less super absorbent materials compared with the deeper layers causing liquids to pass to the deeper layers providing more efficient absorption.
